(19) United States
(12) Patent Application Publication
(10) Pub. No.: US 2021/0100438 A1
(43) Pub. Date: Apr. 8, 2021

(54) ENDOSCOPES AND METHODS OF USE

(71) Applicant: Covidien LP, Mansfield, MA (US)

(72) Inventors: Weijiang Ding, Shanghai (CN); Shenghua Yang, Shanghai (CN); Yancong Lu, Shanghai (CN)

(21) Appl. No.: 16/497,082

(22) PCT Filed: Mar. 24, 2017
(86) PCT No.: PCT/CN2017/078143

§ 371 Date: Sep. 24, 2019

(51) Int. Cl. A61B 1/05; A61B 1/00; A61B 1/06

(52) U.S. Cl. CPC A61B 1/05; A61B 1/00096; A61B 1/0684; A61B 1/00066; A61B 1/00009; A61B 1/0676

(57) ABSTRACT

An endoscope includes a handle, an elongated body, an image sensor, a lens, a light source disposed within a distal portion of the elongated body, a processor, and a controller. The processor is disposed in electrical communication with the image sensor and the light source, and is configured to analyze at least one characteristic of a first image captured by the image sensor. The controller is disposed in electrical communication with the processor, and is configured to supply current to the light source based on the at least one characteristic of the first image analyzed by the processor.

### TECHNICAL FIELD

The present disclosure relates to endosurgical devices, systems and methods for observing internal features of a body during minimally invasive surgical procedures.

### BACKGROUND

Endoscopes are introduced through an incision or a natural body orifice to observe internal features of a body. Conventional endoscopes include a light transmission pathway, including a fiber guide, for transmitting light from an external light source through the endoscope to illuminate the internal features of the body. Conventional endoscopes also include an image retrieval pathway with an optical fiber for transmitting images of these internal features back to an eyepiece or external video system for processing and display on an external monitor. Since the images captured through an optical lens disposed near a distal tip are transferred through the optical fiber to an image sensor near a proximal end of the endoscope, inefficiencies may occur.

To help overcome these inefficiencies relating to the image transfer, a light source and micro camera including an image sensor may be used near the distal tip of the endoscope. However, the production of heat when the light source emits light may result in a loss of functionality and longevity of the light source, for instance. Accordingly, it may be beneficial to help minimize or dissipate the heat emitted from the light source to help optimize the functionality of the light source.

### SUMMARY

The present disclosure relates to an endoscope including a handle and an elongated body extending distally from the handle. The elongated body includes a distal portion terminating at a distal end. An image sensor is disposed within the distal portion of the elongated body and is configured to capture a plurality of images. A lens is disposed adjacent the distal end of the elongated body. A light source is disposed within the distal portion of the elongated body and is configured to illuminate tissue. A processor is disposed in electrical communication with the image sensor and the light source, and is configured to analyze at least one characteristic of a first image of the plurality of images captured by the image sensor. A controller is disposed in electrical communication with the processor, and is configured to supply current to the light source based on the at least one characteristic of the first image of the plurality of images analyzed by the processor.

In disclosed embodiments, the controller is configured to supply current to the light source based at least one characteristic of a second image of the plurality of images captured by the image sensor.

It is further disclosed that the at least one characteristic of the first image may be average gray scale, and the at least one characteristic of the second image may be average gray scale.

In disclosed aspects, the light source may be configured to illuminate tissue when an exposure of the image sensor is open. It is disclosed that the exposure of the image sensor may be configured to be open from about 1/100 seconds to about 1 second before the exposure is closed.

In aspects of the present disclosure, the light source may include one or more (e.g., four) light-emitting diodes.

In disclosed embodiments, the controller is disposed within the handle.

It is further disclosed that the endoscope may include a high thermal conductivity layer disposed adjacent the distal end of the elongated body. In embodiments, the high thermal conductivity layer includes graphene and may have a thickness of from about 0.02 mm to about 0.5 mm.

The present disclosure also relates to a method of using an endoscope. The method includes positioning a distal end of an endoscope adjacent tissue, where the endoscope includes a handle, an elongated body extending distally from the handle and including a distal portion terminating at a distal end. The endoscope also includes an image sensor disposed within the distal portion of the elongated body, a light source disposed within the distal portion of the elongated body, a processor disposed in electrical communication with the image sensor and the light source, and a controller disposed in electrical communication with the processor. The method further includes capturing a first image using the image sensor, analyzing the first image using the processor to obtain at least one characteristic, and supplying a first amount of current to the light source from the controller based on the at least one characteristic of the first image. The method further includes capturing a second image using the image sensor, analyzing the second image using the processor to obtain at least one characteristic, and supplying a second amount of current to the light source from the controller based on the at least one characteristic of the second image.

In disclosed embodiments, the first amount of current is different from the second amount of current.

It is further disclosed that supplying a first amount and a second amount of current to the light source from the controller may be based on average gray scale of the first image and the second image, respectively.

In disclosed aspects, the method may further include opening and closing an exposure of the image sensor, and illuminating tissue using the light source while the exposure of the image sensor is open. In embodiments, the method also includes closing an exposure of the image sensor after the exposure has been open from about 1/100 seconds to about 1 second.

The present disclosure also relates to an endoscope including a handle and an elongated body. The elongated body defines a longitudinal axis and includes a distal portion terminating at a distal end. The endoscope also includes an image sensor disposed within the distal portion of the elongated body and configured to capture a plurality of images, a lens disposed adjacent the distal end of the elongated body, a light source substrate disposed within the distal portion of the elongated body and defining a second axis disposed perpendicularly to the longitudinal axis, and a first light source disposed within the distal portion of the elongated body and configured to illuminate tissue. The first light source may be disposed at a first angle from about 15° to about 45° with respect to the second axis.

In disclosed embodiments, the first angle may be about 30°. Is also disclosed that the endoscope may include a second light source and a third light source disposed within the distal portion of the elongated body. The second light source and the third light source may be disposed at the first angle with respect to the second axis.

It is further disclosed that the endoscope may include a freeform lens disposed in mechanical cooperation with the first light source. In embodiments, the freeform lens and the first light source are configured to provide illumination to only the tissue within a focal range of the image sensor. Further, the freeform lens may be coated with an antireflective film.

According to aspects of the disclosure, the endoscope may include a reflector cup disposed in mechanical cooperation with the first light source. In embodiments, the reflector cup may be coated with a reflective film.

It is disclosed that the first light source may include a light-emitting diode. It is further disclosed that the endoscope may include a processor disposed in electrical communication with the image sensor and the light source. In embodiments, the endoscope includes a controller disposed in electrical communication with the processor, and the controller is configured to supply current to the light source. It is disclosed that the controller may be configured to supply current to the light source when an exposure of the image sensor is open.

It is further disclosed that the endoscope may include a high thermal conductivity layer disposed adjacent the distal end of the elongated body. In embodiments, the high thermal conductivity layer includes graphene, Diamond-like Carbon (DLC), or graphite and has a thickness of from about 0.02 mm to about 0.5 mm.

The present disclosure also relates to an endoscope including a handle and an elongated body extending distally from the handle. The elongated body defines a longitudinal axis and includes a distal portion terminating at a distal end. The elongated body includes an outer shaft. The endoscope also includes an image sensor disposed within the distal portion of the elongated body and configured to capture a plurality of images, a lens disposed adjacent the distal end of the elongated body, a light source disposed within the distal portion of the elongated body and configured to illuminate tissue, and a heat barrier disposed at least partially within the distal portion of the elongated body and configured to block a thermal path from the light source to the outer shaft.

In disclosed embodiments, the heat barrier may be cylindrical. It is disclosed that the endoscope also includes an inner shaft extending at least partially through the elongated body, and that the hear barrier is positioned radially outward of the inner shaft. In embodiments, the heat barrier is positioned radially outward of the image sensor. In embodiments, an outer wall of the heat barrier is flush with an outer wall of the outer shaft.

Is aspects of the disclosure, the heat barrier may include an outer wall and a lip extending radially inward from the outer wall. In embodiments, the lip is positioned proximally of the light source. In further embodiments, the lip is positioned in contact with the light source.

In embodiments, the heat barrier includes a first rib at least partially encircling an inner wall of the heat barrier. It is disclosed that the first rib may be positioned proximally of a proximal surface of the light source. It is further disclosed that the heat barrier may include a second rib at least partially encircling the inner wall of the heat barrier, and that the second rib may be positioned distally of a distal surface of the light source.

In disclosed aspects, the heat barrier may include a plurality of point contacts extending from an inner wall of the heat barrier. In embodiments, a first set of point contacts of the plurality of point contacts is positioned proximally of a proximal surface of the light source, a second set of point contacts of the plurality of point contacts is positioned distally of a distal surface of the light source, and a third set of point contacts of the plurality of points contacts extends distally from a distal face of the heat barrier. It is also disclosed that the endoscope may include a protective window disposed distally of the heat barrier and in contact with the third set of point contacts.

In embodiments, the heat barrier is made from a material selected from a group consisting of polyether ether ketone (PEEK), perfluoroalkoxy (PFA), polyamide-imide (PAI), polyphenylene sulfide (PPS), polyethersulfone (PES), polyetherimide (PEI), polysulfone (PSU), and polyimide (PI).

The present disclosure also relates to a method of using an endoscope including positioning a distal end of an endoscope adjacent tissue, where the endoscope includes a handle, an elongated body extending distally from the handle and including a distal portion terminating at a distal end. The endoscope also includes an image sensor disposed within the distal portion of the elongated body, a light source disposed within the distal portion of the elongated body and including a first group of lights and a second group of lights, and a controller disposed in electrical communication with the light source. The method also includes illuminating tissue with the first group of lights, capturing a first image of the tissue illuminated by the first group of lights using the image sensor, turning off the first group of lights, illuminating tissue with the second group of lights, and capturing a second image of the tissue illuminated by the second group of lights using the image sensor.

In disclosed embodiments, capturing the first image of the tissue illuminated by the first group of lights occurs while the second group of lights is not illuminated. It is disclosed that capturing the second image of the tissue illuminated by the second group of lights may occur while the first group of lights is not illuminated.

Embodiments of the disclosed method also include opening and closing an exposure of the image sensor. In embodiments, illuminating tissue with the first group of lights and illuminating tissue with the second group of lights only occurs when the exposure is open. It is further disclosed that the method includes closing an exposure of the image sensor after the exposure has been open from about 1/100 seconds to about 1 second.

In embodiments, the light source includes a third group of lights, and the method further includes illuminating tissue with the third group of lights and capturing a third image of the tissue illuminated by the third group of lights using the image sensor. It is also disclosed that capturing the third image of the tissue illuminated by the third group of lights may occur while the first group of the lights and the second group of lights are not illuminated.

Further details and aspects of various embodiments of the present disclosure are described in more detail below with reference to the appended figures.

### BRIEF DESCRIPTION OF THE DRAWINGS

Embodiments of the present endoscope systems are described herein with reference to the accompanying drawings, wherein:

FIG. 1 is a front, perspective view of an endoscope system of the prior art;

FIG. 2 is front, perspective view illustrating a schematic configuration of the prior art endoscope system of FIG. 1;

FIG. 3 is a side view illustrating a schematic configuration of an optical system of the prior art endoscope system of FIG. 1;

FIG. 4 is a front, perspective view illustrating a schematic configuration of another prior art endoscope system;

FIG. 5 is a perspective, partial cutaway view illustrating a schematic configuration of a distal end of an endoscope of the prior art endoscope system of FIG. 4;

FIG. 6 is a perspective view of an endoscope in accordance with embodiments of the present disclosure;

FIG. 7 is a schematic configuration of an endoscope system in accordance with an embodiment of the present disclosure;

FIG. 8 is a longitudinal cross-sectional view of an endoscope in accordance with an embodiment of the present disclosure;

FIG. 9 is an enlarged view of a distal portion of the endoscope of FIG. 8;

FIG. 10 is a schematic transverse, cross-sectional view of the distal portion of the endoscope of FIGS. 8 and 9;

FIG. 11 is a schematic cross-sectional view of the distal portion of the endoscope of FIGS. 8-10 taken along line 11-11 of FIG. 10;

FIG. 12 is a graph illustrating the relationship between radiance and current for the endoscope of FIGS. 8-11;

FIG. 13 is a flow chart illustrating a method of using the endoscope of FIGS. 8-11;

FIG. 14 is a longitudinal cross-sectional view of an endoscope in accordance with another embodiment of the present disclosure;

FIG. 15 is an enlarged view of a distal portion of the endoscope of FIG. 14;

FIG. 16 is a schematic cross-sectional view of the distal portion of the endoscope of FIGS. 14 and 15;

FIG. 17 is a schematic transverse, cross-sectional view of the distal portion of the endoscope of FIGS. 14-16;

FIGS. 18-21 are graphs illustrating various relationships of a freeform lens of the endoscope of FIGS. 14-17;

FIG. 22 is a perspective view of an endoscope in accordance with another embodiment of the present disclosure;

FIGS. 23 and 24 are perspective, partial cutaway views illustrating two embodiments of a portion of the endoscope of FIG. 22;

FIG. 25 is a longitudinal cross-sectional view illustrating three embodiments of a portion of the endoscope of FIGS. 22-24;

FIG. 26 is a perspective, partial cutaway view of a distal portion of the endoscope of FIGS. 22-25;

FIGS. 27 and 28 are graphs comparing the endoscope of FIGS. 22-26 with other endoscopes;

FIG. 29 is a perspective, partial cutaway view of a distal portion of an endoscope in accordance with another embodiment of the present disclosure;

FIG. 30 is a side, partial cutaway view of the distal portion of the endoscope of FIG. 29;

FIG. 31 is a perspective view of a heat barrier of the endoscope of FIGS. 29-30;

FIG. 32 is a perspective, partial cutaway view of the heat barrier of FIG. 31;

FIG. 33 is a side, partial cutaway view of a distal portion of an endoscope in accordance with another embodiment of the present disclosure;

FIG. 34 is a perspective, partial cutaway view of the distal portion of the endoscope of FIG. 33;

FIG. 35 is a perspective view of a heat barrier of the endoscope of FIGS. 33-34;

FIG. 36 is a perspective, partial cutaway view of the heat barrier of FIG. 35;

FIG. 37 is a perspective, partial cutaway view of a distal portion of an endoscope in accordance with another embodiment of the present disclosure;

FIG. 38 is a perspective view of a heat barrier of the endoscope of FIG. 37;

FIG. 39 is a perspective, partial cutaway view of the heat barrier of FIG. 38;

FIG. 40 is a longitudinal cross-sectional view of an endoscope in accordance with an embodiment of the present disclosure;

FIG. 41 is an enlarged view of a distal portion of the endoscope of FIG. 40;

FIG. 42 is a schematic cross-sectional view of the distal portion of the endoscope of FIGS. 40 and 41;

FIG. 43 is a graph illustrating the relationship between the illumination of the LEDs and the shutter of the endoscope of FIGS. 40-42;

FIG. 44 is a schematic transverse, cross-sectional view of the distal portion of the endoscope of FIGS. 40-42; and

FIGS. 45 and 46 are graphs illustrating the relationship between the illumination of the LEDs and the shutter of the endoscope of FIGS. 40-42 and 44.

### DETAILED DESCRIPTION OF EMBODIMENTS

Embodiments of the presently disclosed endoscopes and method of use are described in detail with reference to the drawings, in which like reference numerals designate identical or corresponding elements in each of the several views. As used herein, the term “distal” refers to that portion of a structure that is farther from a user, while the term “proximal” refers to that portion of a structure that is closer to the user. The term “clinician” refers to a doctor, nurse, or other care provider and may include support personnel. The term “about” shall be understood as a word of approximation that takes into account relatively little to no variation in a modified term (e.g., differing by less than 2%).

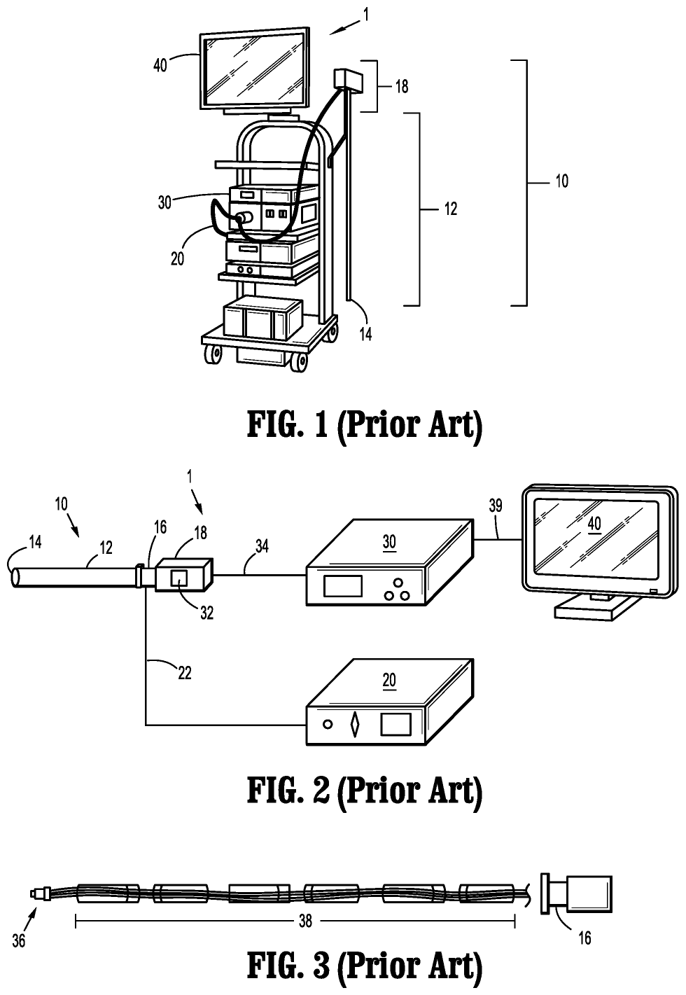

Referring initially to FIGS. 1-3, a prior art endoscope system 1 includes an endoscope 10, a light source 20, a video system 30, and a display device 40. The light source 20, such as an LED/Xenon light source, is connected to the endoscope 10 via a fiber guide 22 that is operatively coupled to the light source 20 and to an endocoupler 16 disposed on, or adjacent to, a handle 18 of the endoscope 10. The fiber guide 22 includes, for example, fiber optic cable which extends through the elongated body 12 of the endoscope 10 and terminates at a distal end 14 of the endoscope 10. Accordingly, light is transmitted from the light source 20, through the fiber guide 22, and emitted out the distal end 14 of the endoscope 10 toward a targeted internal feature, such as tissue or an organ, of a body of a patient. As the light transmission pathway in such a configuration is relatively long, for example, the fiber guide 22 may be about 1.0 m to about 1.5 m in length, only about 15% (or less) of the light flux emitted from the light source 20 is outputted from the distal end 14 of the endoscope 10.

The video system 30 is operatively connected to an image sensor 32 mounted to, or disposed within, the handle 18 of the endoscope 10 via a data cable 34. An objective lens 36 is disposed at the distal end 14 of the elongated body 12 of the endoscope 10 and a series of spaced-apart, relay lenses 38, such as rod lenses, are positioned along the length of the elongated body 12 between the objective lens 36 and the image sensor 32. Images captured by the objective lens 36 are forwarded through the elongated body 12 of the endoscope 10 via the relay lenses 38 to the image sensor 32, which are then communicated to the video system 30 for processing and output to the display device 40 via cable 39.

The image sensor 32 is located within, or mounted to, the handle 18 of the endoscope 10, which can be up to about 30 cm away from the distal end 14 of the endoscope 10. Due to this relatively long distance, there is loss of image information in the image retrieval pathway as it is difficult to get a high quality image at every point along the whole working distance of the relay lenses 38. Moreover, due to light loss on the relay lenses 38, the objective lens 36 cannot include a small aperture. Therefore, the depth of field is limited and a focusing module (not shown) is typically utilized in the endocoupler 16 to set the objective lens 36 to a desired focal point, which a clinician adjusts when moving the endoscope 10 during a surgical procedure. Also, rotation of the fiber guide 22 will also rotate the relay lenses 38, which changes the viewing angle during use, and the fiber guide 22 also tends to fall due to the force of gravity. Accordingly, a clinician needs to adjust and/or hold the fiber guide 22 during use to keep the view stable, which is inconvenient during operation.

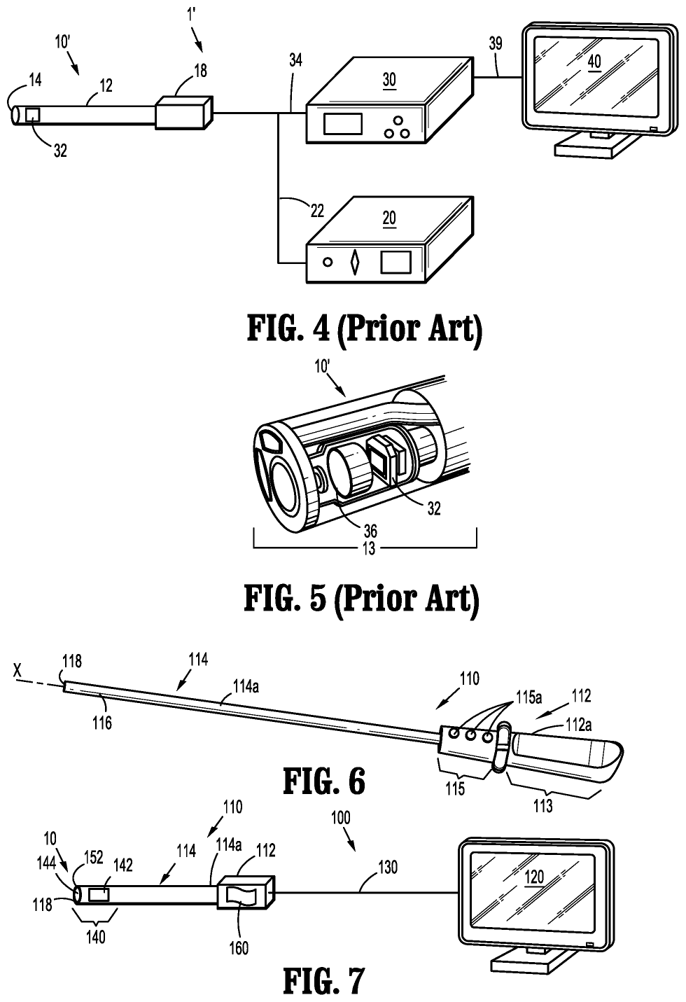

As shown in FIGS. 4 and 5, another prior art endoscope system 1′, which is substantially similar to endoscope system 1 and therefore will only be described with respect to the differences therebetween, includes the image sensor 32 in a distal portion 13 of the elongated body 12 of the endoscope 10′ such that the image retrieval pathway between the objective lens 36 and the image sensor 32 is shorter than that of the endoscope system 1. The endoscope system 1′ adopts the same light transmission pathway as that of the endoscope system 1 (e.g., from the light source 20 and through the fiber guide 22), and thus light consumption on transmission is still large. However, the fiber guide 22 may be integrated with the data cable 34, thereby making the endoscope 10′ easier to operate as a clinician does not need to adjust the fiber guide 22 during use.

Referring now to FIGS. 6 and 7, an endoscope system 100 according to some embodiments of the present disclosure includes an endoscope 110, a display 120, and a cable 130 connecting the endoscope 110 and the display 120. A camera 140, a light source 150, and an integrated processor 160 are contained within the endoscope 110.

The endoscope 110 includes a handle 112 and an elongated body 114 having a cylindrical wall 114a extending distally from the handle 112 and defines a longitudinal axis “x.” The elongated body 114 includes a distal portion 116 terminating at a distal end or tip 118. The handle 112 includes a handle housing 112a including a grip portion 113 for handling by a clinician, and a control portion 115 including actuating elements 115a (e.g., buttons, switches etc.) for functional control of the endoscope 110.

With reference to FIGS. 6 and 7, the camera 140 is disposed within the elongated body 114 of the endoscope 110. The camera 140 includes an image sensor 142 disposed within the distal portion 116 of the elongated body 114 at a location proximal of a lens 144 that is positioned at the distal end 118 of elongated body 114. The image sensor 142 may be a charge-coupled device (CCD), a complementary metal-oxide-semiconductor (CMOS), or a hybrid thereof. In embodiments, the image sensor 142 is a highly sensitive, backside illuminated sensor (BSI). In embodiments, the lighting flux required by the image sensor 142 may be up to about 20 lumens (lm).

As the image retrieval pathway is shortened over that of traditional endoscope systems (e.g., FIG. 1) and the need for relay lenses is eliminated, the depth of field can be expanded and optimized. Accordingly, the lens 144 may include a depth of field from about 20 mm to about 110 mm with optimized image quality and a field-of-view of about 100 degrees. In embodiments, the lens 144 is a focus free lens. As compared to traditional endoscopes, a focus free lens relies on depth of field to produce sharp images and thus eliminates the need to determine the correct focusing distance and setting the lens to that focal point. Accordingly, the aperture of the lens 144 can be relatively small, taking up less space at the distal end 118 of the elongated body 114. In embodiments, the outer diameter of the lens 144 is up to about 6 mm.

The light source 150 is disposed at the distal end 118 of the endoscope 110. Light source 150 includes one or more high efficiency light emitting elements 152, such as light-emitting diodes (LED) arranged in an annular ring around the lens 144 to ensure adequate and even light distribution. In embodiments, the light emitting elements 152 have a luminous efficacy of up to about 80 lm/W (lumen/watt). As compared to traditional endoscopes, the light source of the present disclosure reduces or eliminates the need for the use of an external light source and fiber guide, which can lower the cost of the endoscope system, simplify the endoscope system structure, and reduce light consumption and/or light distortion during light transmission. Although light emitting elements 152 may be efficient and produce less heat than other types of lighting, light emitting elements 152 still produce some heat, which can degrade the quality of the image, for instance.

Thus, managing and minimizing the heat produced by light emitting elements 152 may be beneficial. Heat generation may be managed, for example, by controlling the luminous efficacy of the light emitting elements 152 and the lighting flux required by the image sensor 142. In embodiments, the endoscope 100 of the present disclosure includes high efficiency LED light emitting elements 152 and a BSI CMOS sensor 142. The BSI CMOS sensor 142 reduces the lighting flux required to get a bright and clear image in a desired body cavity over image sensors utilized in traditional endoscopes. Accordingly, in embodiments where, for example, about 20 lm of lighting flux is required, such as within an abdomen of a patient, the power consumption of LED light emitting elements 152 having a luminous efficacy of about 80 lm/W will be about 0.25 W (20 lm/80 lm/W=0.25 W). As about 80% of the power consumption of an LED is typically turned into heat, an LED light emitting element 152 with 0.25 W power consumption would generate no more than about 0.2 W of heat, which is a small amount of heat that may be controlled by a passive thermal system, for example.

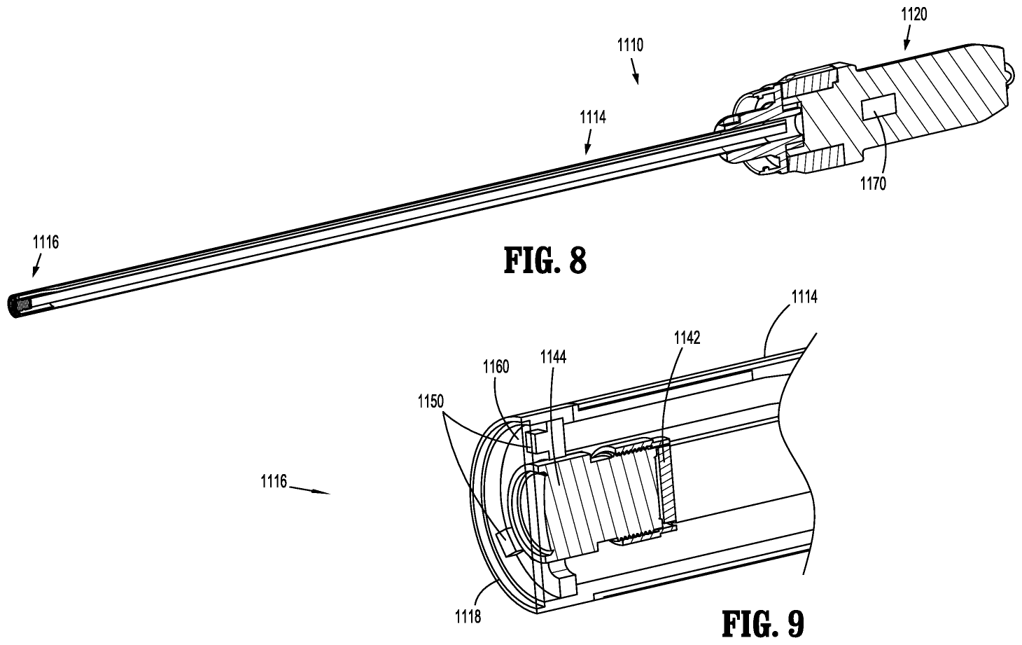

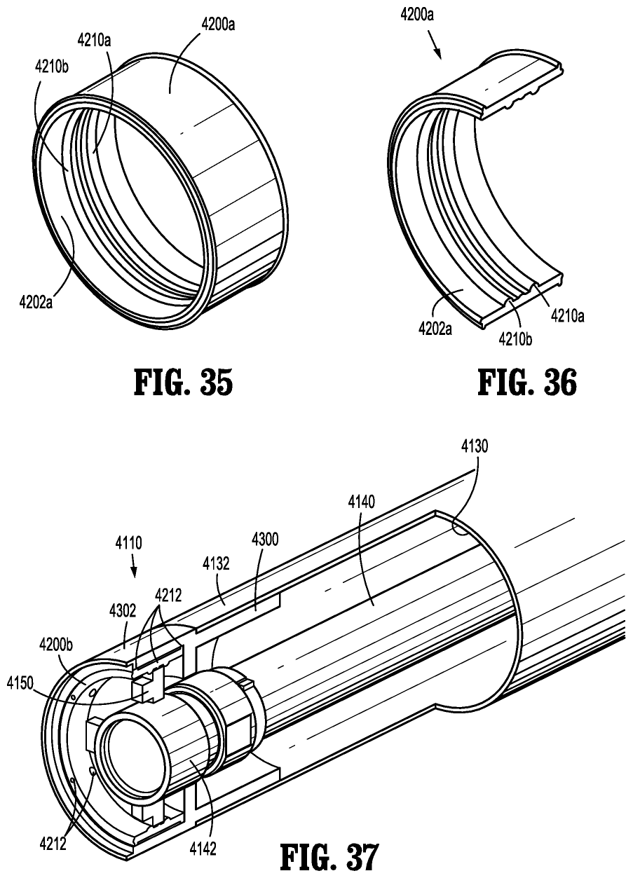

Various endoscopes and methods to manage, reduce and/or dissipate the heat output from the light source are disclosed in FIGS. 8-46. Endoscopes described herein actively and/or passively minimize and/or dissipate the heat produced by its light source. Various endoscopes and methods to observe internal features of a body during minimally invasive surgical procedures which endoscopes include a therapeutic unit, and methods of using these endoscopes to treat tissue are disclosed in corresponding International Patent Application Serial No. PCT/CN2017/078142, filed on Mar. 24, 2017, the entire contents of which are incorporated by reference herein. Other endoscopes that include a passive thermal control system are disclosed in U.S. Patent Application Publication No. 2016/0007833, filed on Jun. 3, 2015, the entire contents of which being incorporated by reference herein. Several types of active and passive thermal control systems are described below and may be used in isolation or in combination with one other.

With particular reference to FIGS. 8-13, an embodiment of an endoscope is shown and is generally referenced by character 1110. Endoscope 1110 utilizes an illumination control technique configured to actively minimize the amount of heat produced by its light source 1150.

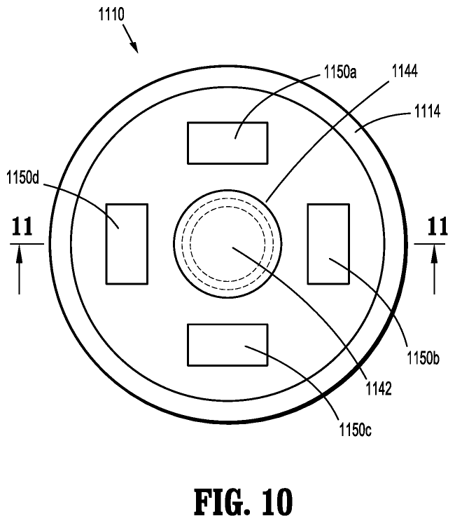

Endoscope 1110 is shown in FIGS. 9-11 and includes a handle 1120 having a controller 1170, and an elongated portion 1114 extending distally from the handle 1120. A distal portion 1116 of the elongated portion 1114 includes an image sensor 1142, a lens 1144, a lens barrel 1146, a protective window 1147, light source (e.g., LED light emitting elements) 1150, a processor 1160, a sensor substrate 1180, and a light source substrate 1190. Distal portion 1116 of elongated portion 1114 terminates in a distal end 1118. In the embodiment illustrated in FIG. 10, light source 1150 includes four LED light emitting elements 1150a, 1150b, 1150c, and 1150d; while four lights emitting elements are illustrated, it is contemplated and within the scope of the present disclosure for more or fewer LED light emitting elements to be used in connection with endoscope 1110. Additionally, LED light emitting elements 1150a, 1150b, 1150c, and 1150d may be any combination of white, red, green and blue light emitting elements, for example.

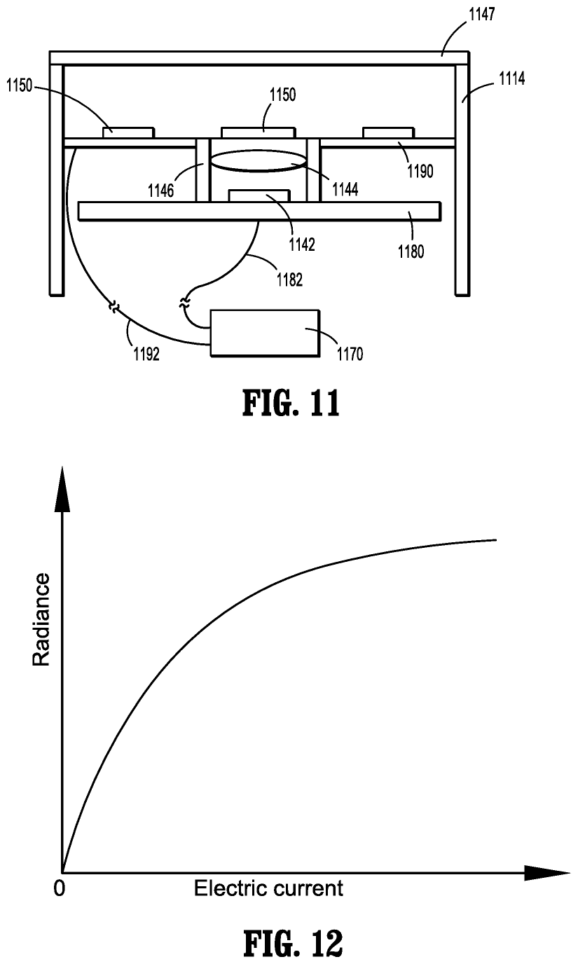

With particular reference to FIGS. 10 and 11, LED light emitting elements 1150a-1150d of light source 1150 are positioned radially outwardly of lens 1144, and are engaged with (e.g., affixed to) light source substrate 1190, which is disposed distally of lens 1144. Sensor substrate 1180 is positioned proximally of lens 1144, and lens barrel 1146 extends distally from image sensor 1142 and from sensor substrate 1180. Lens 1144 is disposed within lens barrel 1146. Image sensor 1142 is engaged with or connected to (e.g., affixed to) sensor substrate 1180. Processor 1160 (FIG. 9) is engaged with or connected to light source 1150 and is in electrical communication with controller 1170.

Controller 1170 is positioned proximally of sensor substrate 1180, and is electrically connected to sensor substrate 1180 and light source substrate 1190 via cables 1182 and 1192, respectively. The engagement between sensor substrate 1180 and image sensor 1142 results in an electrical connection between controller 1170 and image sensor 1142, and the engagement between light source substrate 1190 and light source 1150 results in an electrical connection between controller 1170 and light source 1150.

Controller 1170 controls light source 1150 and image sensor 1142. Controller 1170 may be disposed within handle 1120, and is electrically coupled to processor 1160, sensor substrate 1180 and light source substrate 1190 via at least one cable.

With further regard to processor 1160, it is envisioned that processor 1160 is designed for master control of the described endoscope systems. The processor 1160 is an integrated circuit and may include a system controller, various subsystems, such as an imaging subsystem and a high definition video processing subsystem, and peripherals, such as input/output (I/O) interfaces for controlling data transmission to and/or from external devices, such as the image sensor 1142, the light source 1150, actuating elements in a control portion of the handle 1120, and a display device. The processor 1160 is also responsible for the configuration and control of memory. In embodiments, the processor 1160 is a system-on-chip (SoC). Compared to traditional hardware architecture, the power consumption of a SoC is low resulting in less heat generation. Accordingly, thermal control of the endoscope 1110 is benefitted from a high level integrated, low power consumption SoC processor 1160.

In embodiments, the processor 1160 is configured and designed to capture Full HD raw data from a camera and to transmit the data to the imaging subsystem for video processing, including, for example, color conversion, defect correction, image enhancement, H3A (Auto White Balance, Auto Exposure, and Auto Focus), and resizer. The data is then transmitted to the high definition video processing subsystem for wrapping of the processed data, and finally to an HDMI output for image display on a display device. The hardware modules may be tailored to control power consumption. In embodiments, some hardware functional blocks, such as a high definition video image co-processor, and some peripherals, such as Ethernet and some I/O interfaces, may be disabled. Such system software optimization of the video pipeline results in lower resource requirements and the tailored hardware modules optimize power consumption for thermal control.

Endoscope 1110 is configured to help ensure a minimal amount of heat is produced by light source 1150. Specifically, image sensor 1142 is set with a constant exposure time, which is determined in part by the frequency of shooting an image, and the quality of the images. It is envisioned that the exposure time may be from about 1/100 seconds to about 1 second, and that the interval between exposures equals the reciprocal of the frame frequency minus exposure time and it may be from about 1/100 seconds to about 1 second. Light source 1150 is configured to only provide illumination (and thus produce heat) when the exposure of image sensor 1142 is open. Further, the amount of radiance of light source 1150 is determined by analyzing characteristics (e.g., average gray scale, mid value of gray scale, maximum value of gray scale, and minimum value of gray scale) of the previous image that was captured by image sensor 1142. Processor 1160 is configured to analyze characteristics of images, which are then relayed to controller 1170. Based on the characteristics of images received by controller 1170, controller 1170 supplies light source 1150 with an amount of current to supply an appropriate radiance to efficiently maintain the characteristics at an appropriate value. FIG. 12 illustrates a typical relationship between current and radiance.

For instance, when one characteristic being used is the average gray scale of an image, the amount of radiance of light source 1150 is preset at a normal or average value, and a first image is captured by image sensor 1142. Processor 1160 then calculates the average gray scale of the first image and relays this information to controller 1170. According to the average gray scale, controller 1170 supplies light source 1150 with the appropriate amount of current such that the radiance of light source 1150 is adjusted to maintain the value of the average gray scale that was previously calculated.

Additionally, embodiments of endoscope 1110 may include a proximity sensor of a known type that is configured to detect a distance between image sensor 1142 and the object being sensed by image sensor. If the distance between endoscope 1110 and the object being sensed changes, the radiance of light source 1150 may be adjusted by the controller to optimize the radiance and to ensure the average gray scale is maintained at an appropriate value. Thus, controller 1170 receives information from the proximity sensor and/or processor 1160 and adjusts the current output accordingly.

The information received by processor 1160 regarding the average gray scale and proximity, for example, helps allow image sensor 1142 to obtain high quality images by always providing enough radiance to maintain the average gray scale within a predetermined range, for instance. Additionally, this information helps distal portion 1116 of endoscope 1110 maintain a relatively low temperature (as compared to the current being consistently supplied to provide a large amount of radiance), thus prolonging the life of endoscope 1110 and reducing the possibility of unnecessarily heating tissue. Additionally, to further help maintain the relatively low temperature of distal portion 1116 of endoscope 1110, light source 1150 is configured to only provide illumination (and thus produce heat) when the exposure of image sensor 1142 is open, as discussed above.

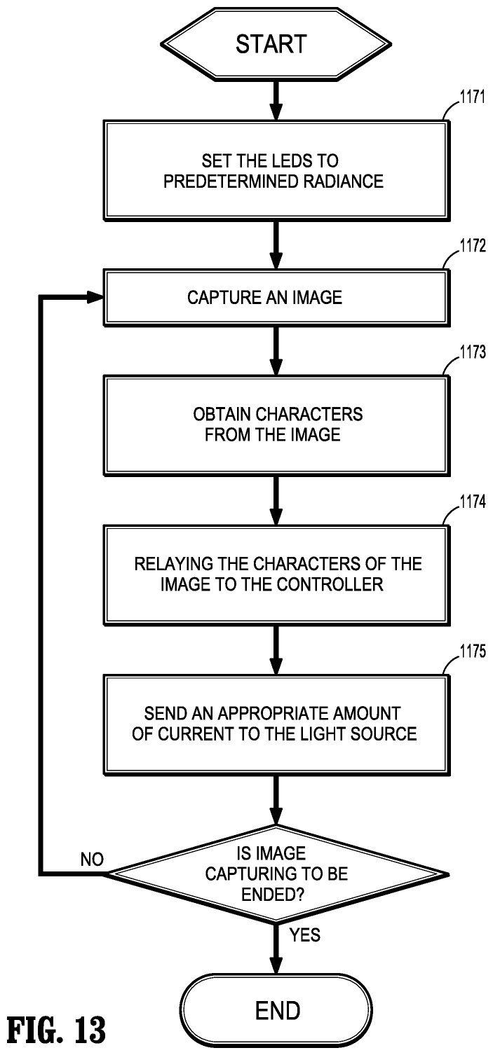

With particular reference to FIG. 13, a flowchart illustrating the various steps of minimizing the current delivered to light source 1150 is shown. A first step 1171 includes setting the radiance of LED or light source 1150 to a predetermined value. A second step 1172 includes capturing an image by image sensor 1142. A third step 1173 includes obtaining at least one characteristic (e.g., average gray scale) from the previously-captured image using processor 1160. A fourth step 1174 includes relaying the at least one characteristic of the image to controller 1170. A fifth step 1175 includes using controller 1170 to send an appropriate amount of current to light source 1150 based on the characteristic of the image. If endoscope 1110 is still being used, each of the second step 1172 through the fifth step 1175 is repeated. Using this method, a large percentage of the radiance, and thus current, is used for imaging which results in much less heat being produced when compared to traditional illumination techniques. This reduction in the production of heat may improve the image quality, increase the life of endoscope 1110, and reduce the chances of unnecessarily heating tissue with distal portion 1116 of endoscope 1110.

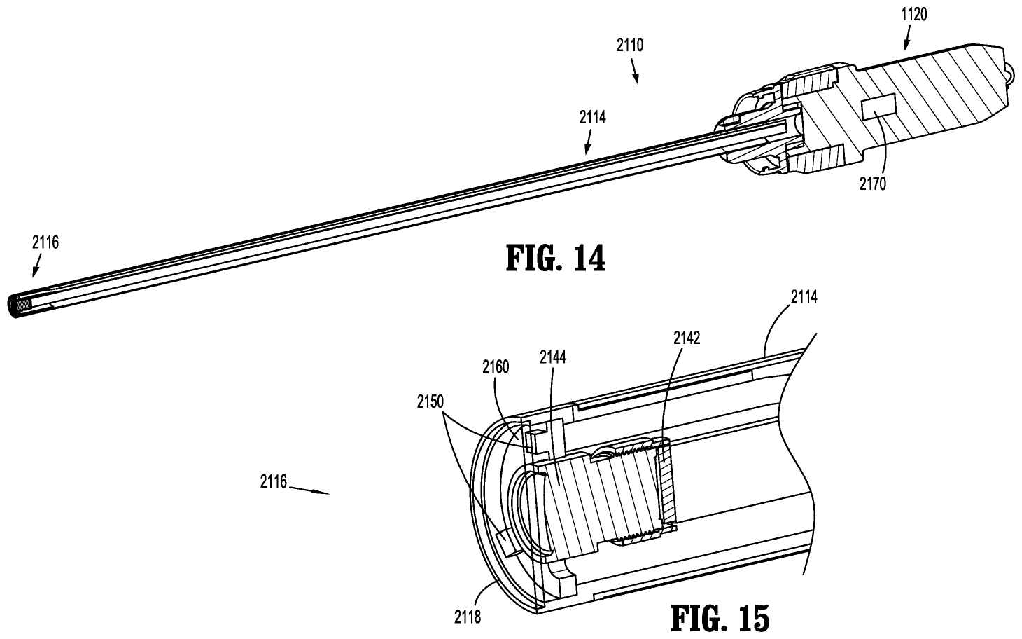

With particular reference to FIGS. 14-21, an embodiment of an endoscope is shown and is generally referenced by character 2110. Endoscope 2110 provides increased illumination efficiency to minimize the amount of heat produced by its light source 2150. In the interest of brevity, some similarities between endoscope 2110 and endoscope 1110 are not discussed in detail. Additionally, various features of endoscope 2110 may be used in connection with endoscope 1110 (and vice versa), described above.

Endoscope 2110 is shown in FIGS. 14-17 and includes a handle 2120, and an elongated portion 2114 extending distally from the handle 2120. A distal portion 2116 of the elongated portion 2114 includes an image sensor 2142, a lens 2144, a lens barrel 2146, a protective window 2147, light source (e.g., LED light emitting elements) 2150, a processor 2160, a sensor substrate 2180, and a light source substrate 2190. Distal portion 2116 of elongated portion 2114 terminates in a distal end 2118.

Additionally, distal portion 2116 of the elongated portion 2114 includes an adhesive 2130 (e.g., a conductive adhesive), reflector cups 2132, and freeform lenses 2134. Adhesive 2130 is disposed between light source 2150 and light source substrate 2190, and helps light source 2150 adhere to light source substrate 2190 at an appropriate angle (as discussed below). Each reflector cup 2132 and freeform lens 2134 is disposed in mechanical cooperation with light source 2150 (e.g., an individual LED light emitting element of light source 2150).

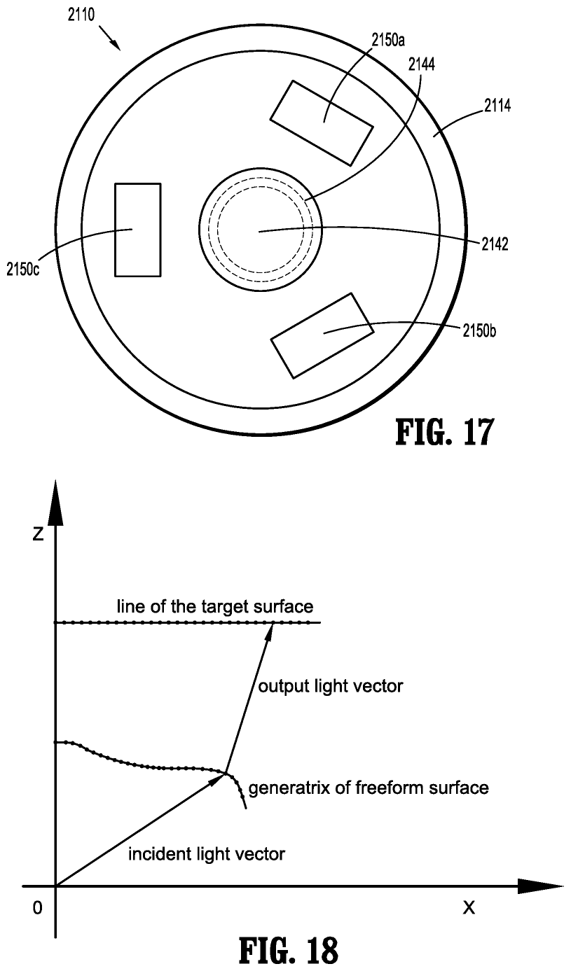

In the embodiment illustrated in FIG. 17, light source 2150 includes three LED light emitting elements 2150a, 2150b, and 2150c; while three LED light emitting elements are illustrated, it is contemplated and within the scope of the present disclosure for more or fewer LED light emitting elements to be used in connection with endoscope 2110. Additionally, LED light emitting elements 2150a, 2150b and 2150c may be any combination of white, red, green and blue light emitting elements, for example.

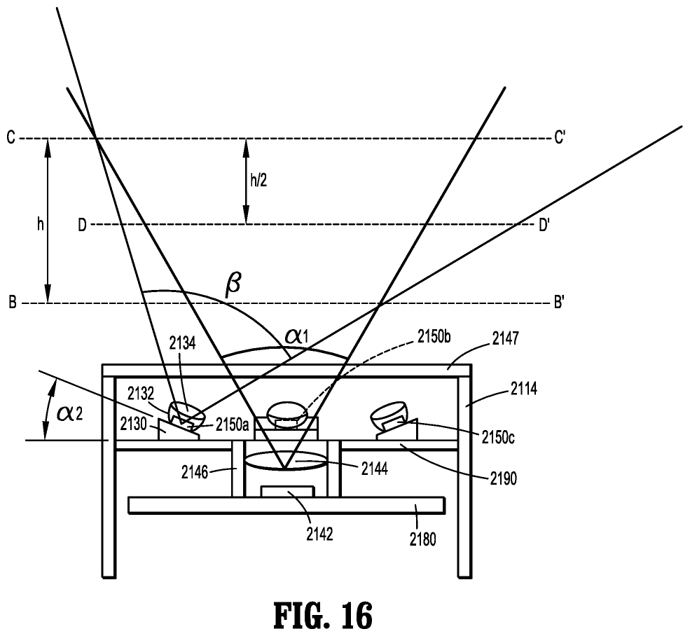

With particular reference to FIGS. 16 and 17, LED light emitting elements 2150a-2150c of light source 2150 are positioned radially outwardly of lens 2144, and are engaged with (e.g., affixed to) light source substrate 2190 via adhesive 2130; light source substrate 2190 is disposed distally of lens 2144. Sensor substrate 2180 is positioned proximally of lens 2144, and lens barrel 2146 extends distally from image sensor 2142 and from sensor substrate 2180. Lens 2144 is disposed within lens barrel 2146. Image sensor 2142 is engaged with or connected to (e.g., affixed to) sensor substrate 2180. In embodiments, processor 2160 (FIG. 15) is engaged with or connected to light source 2150 and is in electrical communication with a controller 2170 disposed within handle 2120.

In embodiments where endoscope 2110 includes controller 2170, controller 2170 is electrically connected to sensor substrate 2180 and light source substrate 2190 via cables, for example. The engagement between sensor substrate 2180 and image sensor 2142 results in an electrical connection between controller 2170 and image sensor 2142, and the engagement between light source substrate 2190 and light source 2150 results in an electrical connection between controller 2170 and light source 2150.

Endoscope 2110 is configured to help ensure a minimal amount of heat is produced by light source 2150 by increasing the illumination efficiency of light source 2150. In particular, the use of reflector cups 2132 (e.g., highly reflective) and freeform lenses 2134 (e.g., antireflective) help provide a high uniformity of illumination and an appropriate illuminating angle, while maintaining a low temperature of distal portion 2116 of elongated portion 2114.

More particularly, light source 2150, reflector cups 2132 and freeform lenses 2134 are configured to focus the illumination of light source 2150 on the tissue that is being imaged by image sensor 2142 such that only tissue within the focal range of image sensor 2142 is illuminated. With reference to FIG. 16, the image angle of image sensor 2142 is indicated as al, the depth of focus ranges from B-B′ to C-C′, with the total length (e.g., the distance between B-B′ and C-C′) indicated as “h,” and with D-D′ representing the middle of the field of focus. The illuminating angle of light emitting element 2150a is indicated as β. Additionally, each light emitting element 2150a-2150c of light source 2150 is positioned at a fixed angle α2 on light source substrate 2190. It is envisioned that angle α2 may be from about 0° to about 45° degrees (e.g., approximately equal to 30°). Accordingly, as shown in FIG. 16, the image angle α1 of image sensor 2142 intersects the illuminating angle β of light emitting element 2150a at focus position B-B′ and at focus position C-C′, thus efficiently encompassing the entire focal range “h.” The other light emitting elements 2150b and 2150c are similarly (or identically) angled such that their illumination angle also efficiently encompasses the entire focal range “h” of image sensor 2142.

With particular reference to FIGS. 18-20, each freeform lens 2134 includes two surfaces. A proximal or first surface 2134a of freeform lens 2134 is disposed closest to light source 2150 and is spherical. When light emitted from light source 2150 passes first surface 2134a, the light propagates along the same direction; the light does not bend as it passes through first surface 2134a of freeform lens 2134.

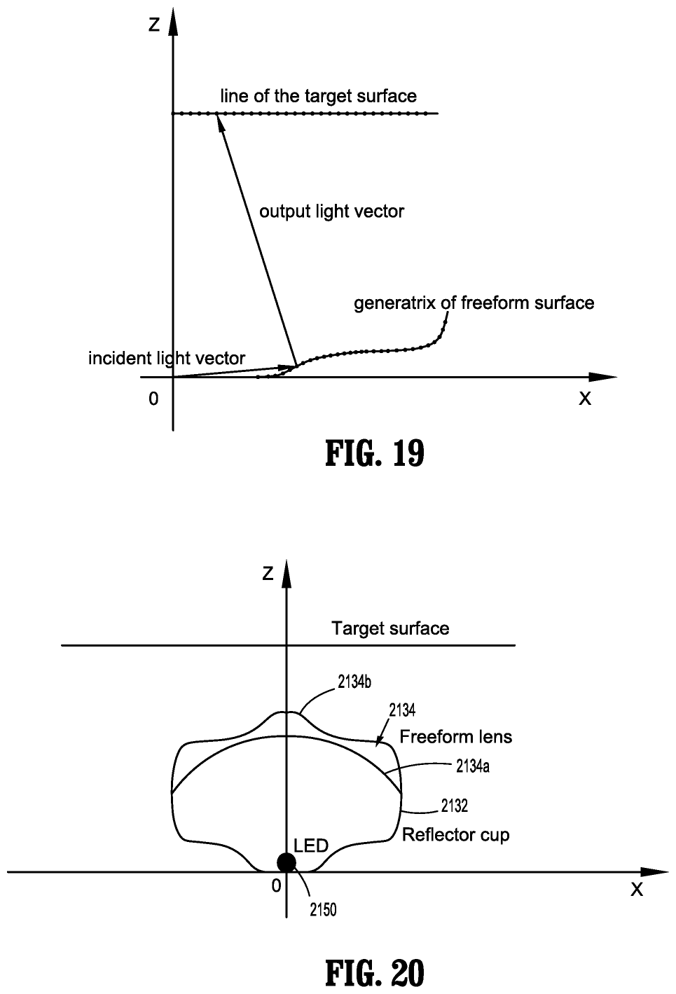

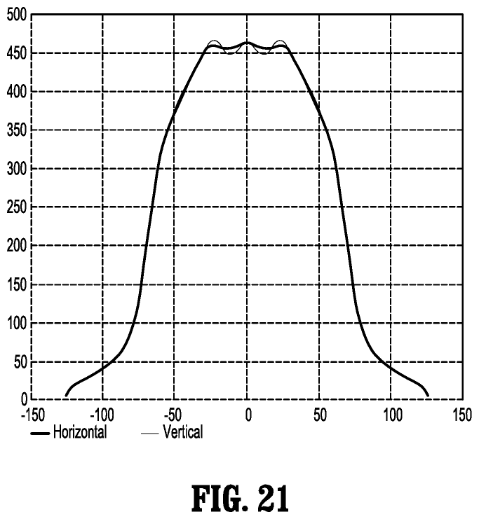

A distal or second surface 2134b of freeform lens 2134 includes a freeform surface designed to refract the light to an expected position. An example of the curvature of second surface 2134b is shown in FIGS. 18-20. To determine the particular curvature or generatrix of second surface 2134b, light source 2150 is set at an original point “O,” and each point of the generatrix is positioned such that light energy of each point has the same light energy of a corresponding point of a line of the target surface. Since each point on the generatrix corresponds to one point on the target line (FIGS. 18 and 19), the propagating direction of every light ray emitted from light source 2150 can be determined. Based on the incident vector and the output vector, short freeform lines on every point can be determined. Finally, the generatrix of second surface 2134b can be obtained by connecting each short line. The resulting generatrix of second surface 2134b of freeform lens 2134 results in a relatively uniform illumination of the target surface. An illustrative example of the intensity (W/m²) of the illumination along the horizontal and vertical directions of different positions (millimeter) is shown in FIG. 21.

Additionally, reflector cups 2132 can also include a freeform surface, which may be determined in a similar manner to that of second surface 2134b of freeform lens 2134.

Further, to help ensure a minimal amount of light is reflected back toward distal portion 2116 of elongated portion 2114, freeform lenses 2134 and protective window 2147 may be coated with a high antireflective film (e.g. MgF₂, TiO₂, ZnSe, ZnS), and reflector cups 2132 may be coated with a high reflective film (e.g. Al, Ag, Au).

Additionally, to further help ensure a minimal amount of heat is produced by light source 2150, light source 2150 may be configured to only provide illumination (and thus produce heat) when the exposure of image sensor 2142 is open. In such embodiments, controller 2170 is used to send current (and thus radiance) to light source 2150 only when the exposure of image sensor 2142 is open, thus reducing the total amount of heat produced.

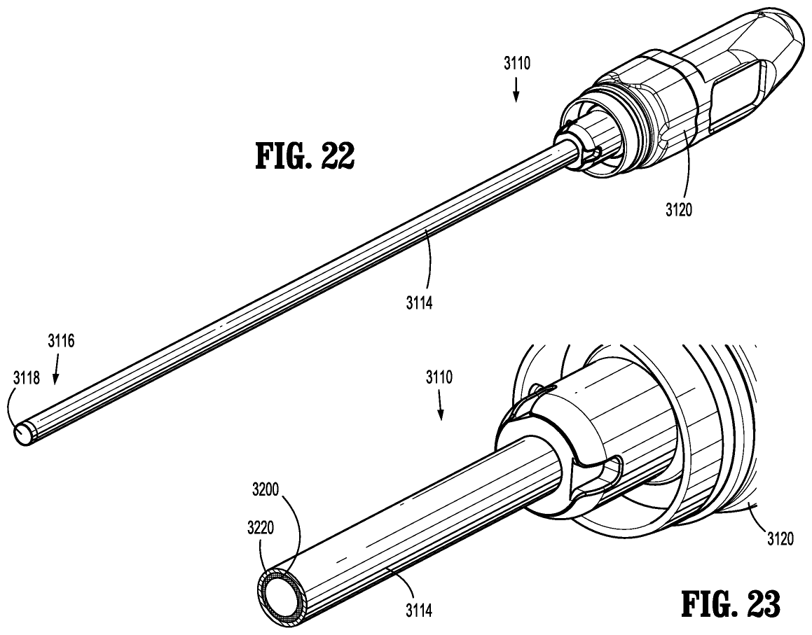

With particular reference to FIGS. 22-28, an embodiment of an endoscope is shown and is generally referenced by character 3110. Endoscope 3110 includes a passive thermal control system to help reduce the temperature of its distal portion 3116 and distal end 3118. In the interest of brevity, some similarities between endoscope 3110 and endoscopes 1110 and 2110 are not discussed in detail. Additionally, various features of endoscope 3110 may be used in connection with endoscopes 1110 and/or 2110 (and vice versa), described above.

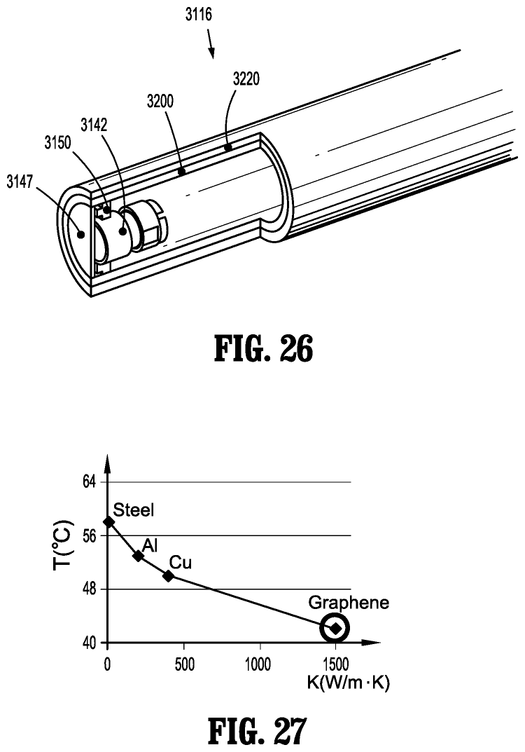

Endoscope 3110 includes a handle 3120 and an elongated portion 3114 extending distally from the handle 3120. The distal portion 3116 of the elongated portion 3114 includes an image sensor 3142, a light source 3150 (e.g., LED light emitting elements) and a protective window 3147, for example, such as those described above with reference to endoscopes 1110 and 2110 (and see FIG. 26). Endoscope 3110 also includes a high thermal conductivity layer 3200 to help reduce the temperature at and near distal end 3118.

Generally, high thermal conductivity layer 3200 extends between the distal portion 3116 of the elongated portion 3114 and a part of the elongated portion 3114 closer to the handle 3120. High thermal conductivity layer 3200 is configured to conduct the heat produced by the light source 3150 proximally toward the handle 3120 of endoscope 3110. This conduction of heat utilizes part of the elongated portion 3114 as a heat sink and helps reduce the temperature near the distal end 3118 of the elongated portion 3114, which often has the highest temperature of the endoscope 3110.

In typical endoscopes, an elongated tube 3220 is often made from stainless steel, which has a relatively low thermal conductivity and thus poorly conducts heat away from the distal end of the elongated portion. The high thermal conductivity layer 3200 of endoscope 3110 is made from a material whose K value is greater than 600 W/mK, such as graphene, graphite or Diamond-Like Carbon (DLC).

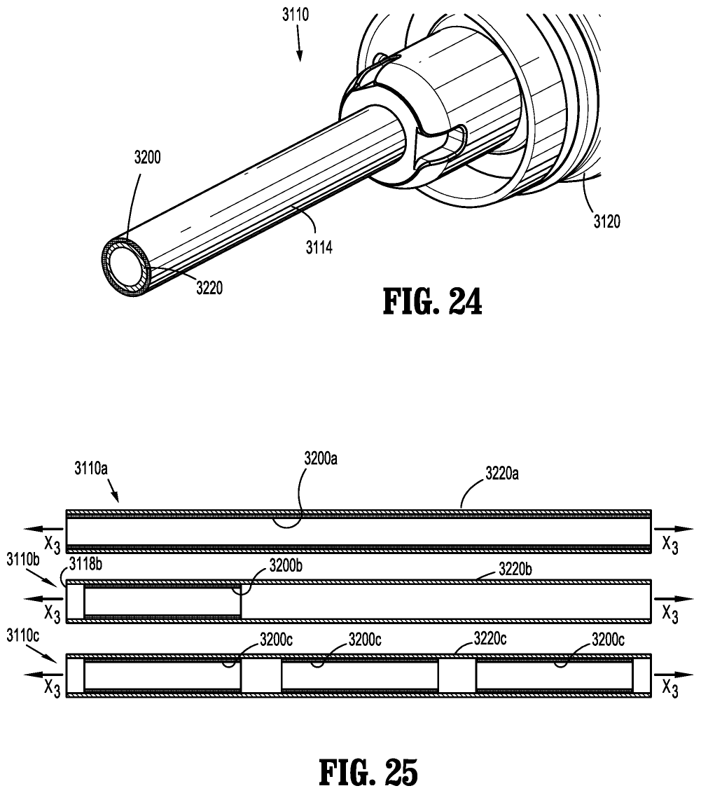

With particular reference to FIGS. 23 and 34, high thermal conductivity layer 3200 may be positioned radially inward of the elongated tube 3220 (FIG. 23), or radially outward of the elongated tube 3220 (FIG. 24). Additionally, in endoscopes including an inner shaft disposed within an outer tube, the high thermal conductivity layer 3200 may be positioned on the inner shaft. The high thermal conductivity layer 3200 may have a thickness of from about 0.02 mm to about 0.5 mm, for instance.

Additionally, and with particular reference to FIG. 25, three embodiments of endoscope 3110 are shown and are indicated as 3110a, 3110b, and 3110c. Each of the three embodiments of endoscope show the high thermal conductivity layer 3200 at a different position along a longitudinal axis x3-x3 of the endoscope 3110a, 3110b, and 3110c. In the first embodiment of endoscope 3110a, high thermal conductivity layer 3200a is disposed along an entirety of a length of the elongated tube 3220a. In the second embodiment of endoscope 3110b, high thermal conductivity layer 3200b is disposed toward a distal end 3118b of the elongated tube 3220b. In the third embodiment of endoscope 3110c, high thermal conductivity layer 3200c is disposed in three separate segments along a length of the elongated tube 3220c. It is envisioned that endoscope 3110 of the present disclosure includes any of these configurations of the high thermal conductivity layer 3200, or any combination thereof. Additionally, it is contemplated and within the scope of the present disclosure for the high thermal conductivity layer 3200 to be otherwise configured with respect to the elongated tube 3220.

Referring now to FIG. 27, a graph showing an illustrative effect of the high thermal conductivity layer 3200 on temperature reduction is shown. The graph illustrates the temperature differences at the distal end 3118 of the endoscope 3110 when different materials are used for the high thermal conductivity layer 3200. As shown, when steel is used for the high thermal conductivity layer 3200, the temperature at the distal end 3118 of the endoscope 3110 is about 58° C. When aluminum (AL) is used for the high thermal conductivity layer 3200, the temperature at the distal end 3118 of the endoscope 3110 is about 52° C. When copper (CU) is used for the high thermal conductivity layer 3200, the temperature at the distal end 3118 of the endoscope 3110 is about 50° C. Finally, when graphene is used for the high thermal conductivity layer 3200, the temperature at the distal end 3118 of the endoscope 3110 is about 42° C. Accordingly, the use of graphene for the high thermal conductivity layer 3200 passively reduces temperature at the distal end 3118 of the endoscope 3110.

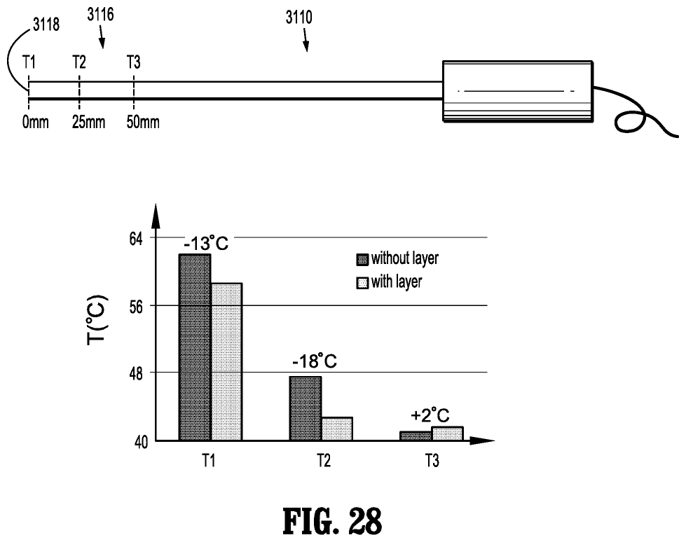

Referring now to FIG. 28, a comparison of typical temperatures along various parts of the distal portion 3116 of the elongated portion 3114 on the endoscope 3110 is shown. The chart compares temperatures at three places along the distal portion 3116 of the endoscope 3110 with and without the high thermal conductivity layer 3200. As shown, the temperature at T1, the distal end 3118 of the endoscope 3110, without a high thermal conductivity layer is about 13° C. higher than the temperate at T1 with the high thermal conductivity layer 3200. Additionally, the temperature at T2, which is 25 mm proximal of the distal end 3118 of the endoscope 3110, without a high thermal conductivity layer is about 18° C. higher than the temperate at T2 with the high thermal conductivity layer 3200. Finally, the temperature at T3, which is 50 mm proximal of the distal end 3118 of the endoscope 3110, without a high thermal conductivity layer is about 2° C. lower than the temperate at T3 with the high thermal conductivity layer 3200. While the temperature at T3 is higher with the high thermal conductivity layer 3200 than without the high thermal conductivity layer, the temperature is about 43° C., which is within an acceptable range for body contact.

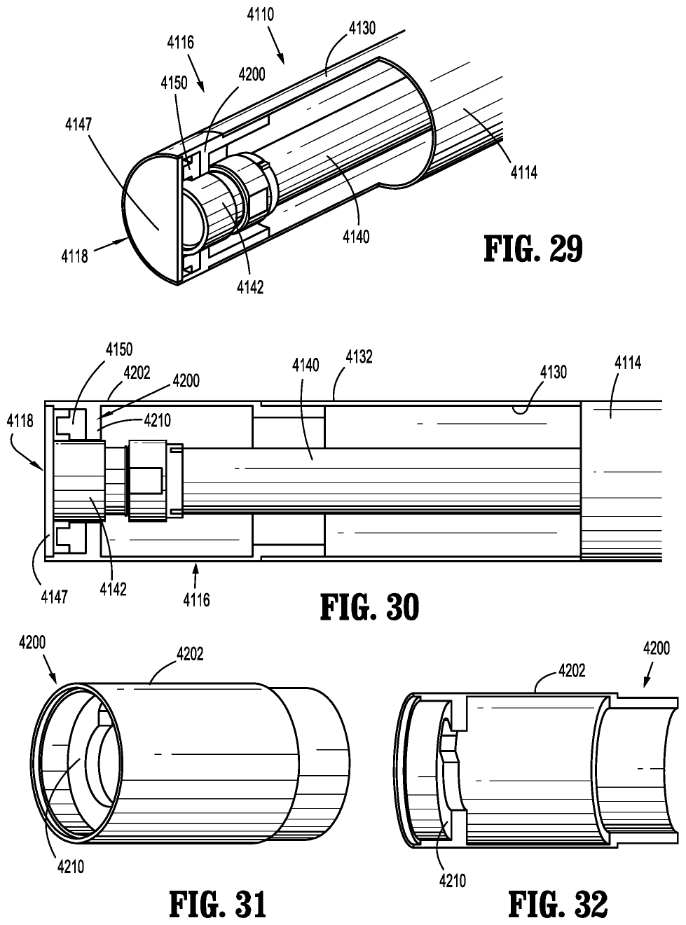

With particular reference to FIGS. 29-39, an embodiment of an endoscope is shown and is generally referenced by character 4110. Endoscope 4110 includes a passive thermal control system to help reduce the temperature of its distal portion 4116 and distal end 4118. In the interest of brevity, some similarities between endoscope 4110 and endoscopes 1110, 2110 and 3110 are not discussed in detail. Additionally, various features of endoscope 4110 may be used in connection with endoscopes 1110, 2110 and/or 3110 (and vice versa), described above.

Endoscope 4110 includes a handle and an elongated portion 4114 extending distally from the handle. The distal portion 4116 of the elongated portion 4114 includes an image sensor 4142, a light source 4150 (e.g., LED light emitting elements) and a protective window 4147, for example, such as those described above with reference to endoscopes 1110, 2110 and 3110. Endoscope 4110 also includes a heat barrier 4200 to help reduce the temperature at and near distal end 4118.

Generally, heat barrier 4200 is disposed at or near the distal end 4118 of the endoscope 4110 and is configured to reduce the amount of heat that reaches an outer shaft 4130 of the endoscope 4110. Further, the heat barrier 4200 is made from a material with a large thermal resistance (discussed in further detail below) and blocks the thermal path from the heat source (e.g., light source 4150) to the outer shaft 4130, for instance.

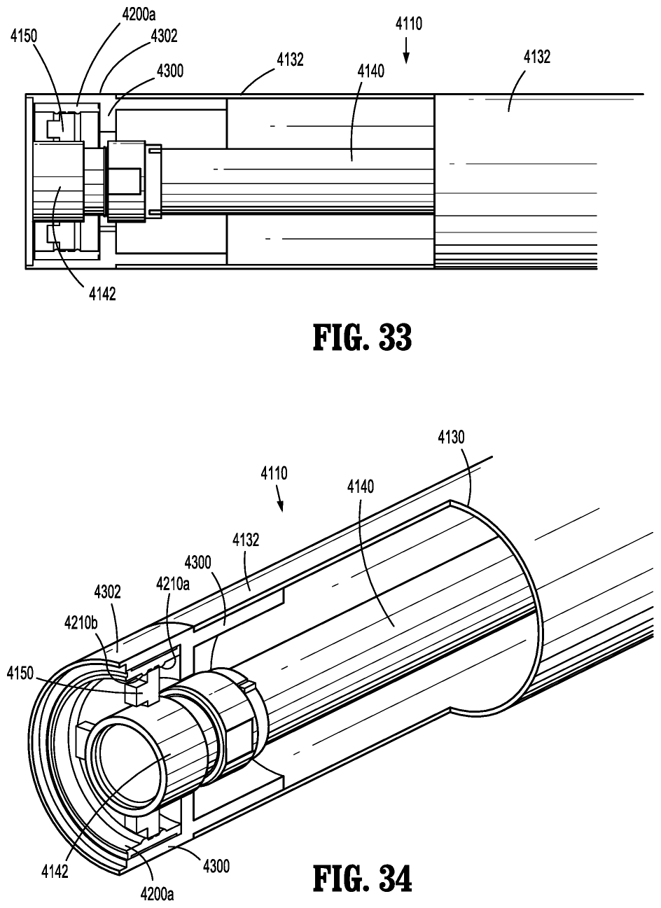

With particular reference to FIGS. 29-32, an embodiment of endoscope 4110 is shown including heat barrier 4200 as a replacement for a distal tip 4300 (the distal tip 4300 is shown in FIGS. 33, 34 and 37, for example). Heat barrier 4200 includes a cylindrical shape that is positioned radially outward of an inner shaft 4140 of endoscope 4110, and radially outward of image sensor 4142. An outer wall 4202 of heat barrier 4200 is radially aligned or flush with an outer wall 4132 of outer shaft 4130.

Further, with particular reference to FIG. 30, heat barrier 4200 includes a lip 4210 extending radially inward from the outer wall 4202. The lip 4210 is positioned proximally of (e.g., in contact with) the light source 4150. Since the light source 4150 produces heat, contact (or near contact) between the heat barrier 4200 and the light source 4150 helps efficiently reduce the temperature of the light source 4150 and thus the distal portion 4116 of the elongated portion 4114 of the endoscope 4110. It is envisioned that heat barrier 4200 includes ribs (similar to ribs 4210a, 4210b discussed below) or point contacts (similar to point contacts 4212 discussed below) to increase its efficiency or strength, for example.

With particular reference to FIGS. 33-36, an embodiment of endoscope 4110 is shown including another embodiment of heat barrier 4200a which is used in connection with the distal tip 4300 of endoscope 4110. Heat barrier 4200a includes a cylindrical shape that is positioned radially outward of inner shaft 4140 of endoscope 4110, radially outward of image sensor 4142, and radially inward of distal tip 4300. An outer wall 4302 of distal tip 4300 is radially aligned or flush with the outer wall 4132 of outer shaft 4130.

Further, and with particular reference to FIGS. 34-36, heat barrier 4200a includes a pair of ribs (or line contacts) including a proximal rib 4210a and a distal rib 4210b extending around or at least partially encircling an inner wall 4202a of heat barrier 4200a. The proximal rib 4210a is positioned proximally of (e.g., in contact with) a proximal surface of the light source 4150, and the distal rib 4210b is positioned distally of (e.g., in contact with) a distal surface of the light source 4150. The contact or proximity between the pair of ribs 4210a, 4210b and the light source 4150 helps properly orient heat barrier 4200a with respect to the light source 4150, and helps ensure an efficient temperature reduction of the light source 4150 due to the contact and/or proximity therebetween, thus providing an efficient temperature reduction of the distal portion 4116 of the elongated portion 4114 of the endoscope 4110.

Referring now to FIGS. 37-39, an embodiment of endoscope 4110 is shown including another embodiment of heat barrier 4200b which is used in connection with the distal tip 4300 of endoscope 4110. Heat barrier 4200b includes a cylindrical shape that is positioned radially outward of inner shaft 4140 of endoscope 4110, radially outward of image sensor 4142, and radially inward of distal tip 4300. An outer wall 4302 of distal tip 4300 is radially aligned or flush with the outer wall 4132 of outer shaft 4130.

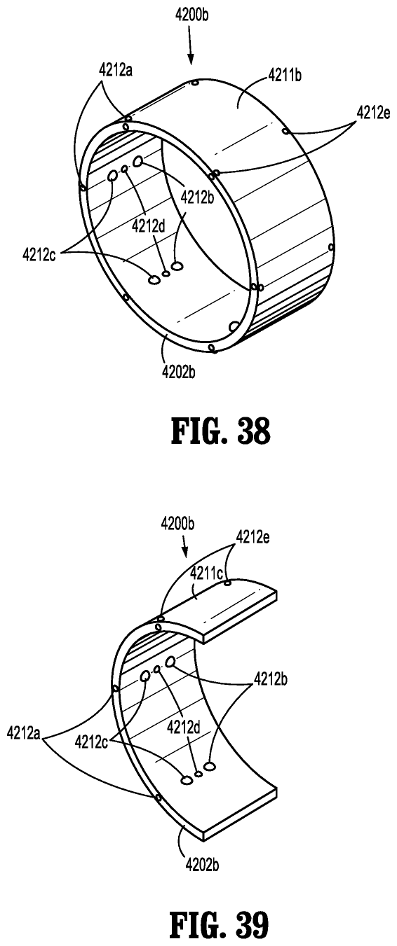

Further, heat barrier 4200b includes a plurality of point contacts 4212 extending from various surfaces of heat barrier 4200b. With reference to FIGS. 38 and 39, a first set of point contacts 4212a is shown extending distally from a distal face 4202b of heat barrier 4200b and are configured to contact and reduce the heat of the protective window 4147 (FIG. 33). Each of a second set of point contacts 4212b and a third set of point contacts 4212c is respectively positioned proximally of (e.g., in contact with) a proximal surface of the light source 4150, and distally of (e.g., in contact with) a distal surface of the light source 4150 and is configured to ensure an efficient temperature reduction of the light source 4150 due to the contact and/or proximity therebetween, for instance. A fourth set of point contacts 4212d may be disposed between the second set of point contacts 4212b and the third set of point contacts 4212c, and a fifth set of point contacts 4212e may be disposed on an outer wall 4211b of heat barrier 4200b. Both of the fourth set of point contacts 4212d and the fifth set of point contacts 4212e may further help reduce the temperature of various features of the endoscope 4110, such as the light source 4150 and the distal tip 4300.

In disclosed embodiments, heat barriers 4200, 4200a, 4200b are made from at least one material having a low thermal conductivity and a high temperature endurance, such as, for example, polyether ether ketone (PEEK), perfluoroalkoxy (PFA), polyamide-imide (PAI), polyphenylene sulfide (PPS), polyethersulfone (PES), polyetherimide (PEI), polysulfone (PSU), or polyimide (PI). In embodiments, the replacement of the distal tip 4300 with heat barrier 4200, may cause the temperature of the outer shaft 4130 to reduce by 50% or more, in some embodiments from about 58.6° C. to about 26.2° C. Additionally, in embodiments, the use of heat barrier 4200a causes the temperature of the outer shaft 4130 to reduce from about 58.6° C. to about 29.3° C. In embodiments, the use of heat barrier 4200b causes the temperature of the outer shaft 4130 to reduce from about 58.6° C. to about 25.6° C.

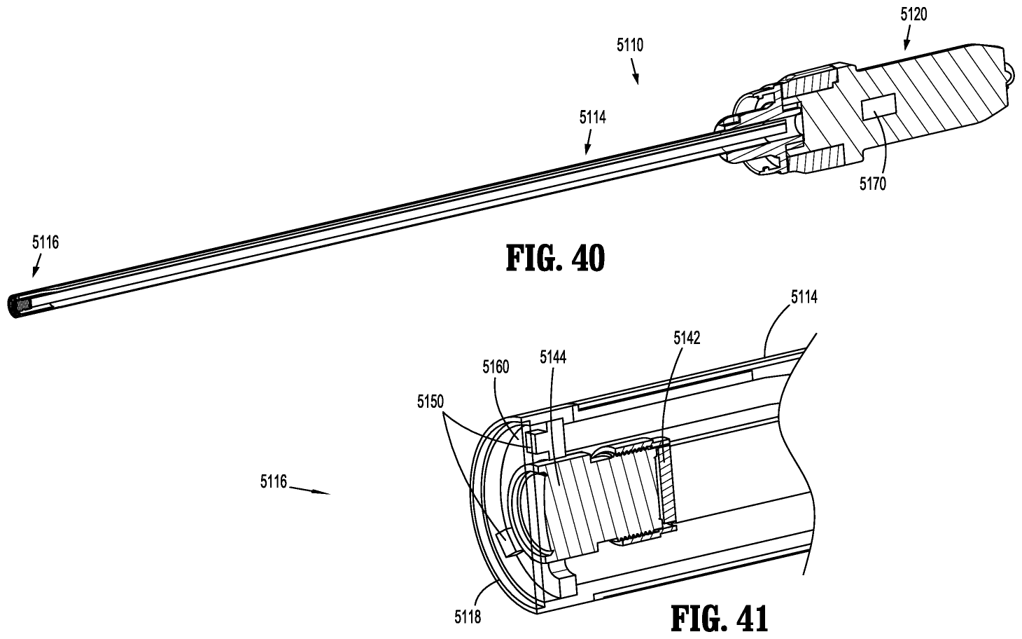

With particular reference to FIGS. 40-46, an embodiment of an endoscope is shown and is generally referenced by character 5110. Endoscope 5110 utilizes an illumination control technique configured to actively minimize the amount of heat produced by its light source 5150. In the interest of brevity, some similarities between endoscope 5110 and endoscopes 1110, 2110, 3110 and 4110 are not discussed in detail. Additionally, various features of endoscope 5110 may be used in connection with endoscopes 1110, 2110, 3110 and/or 4110 (and vice versa), described above.

Endoscope 5110 includes a handle 5120 having a controller 5170, and an elongated portion 5114 extending distally from the handle 5120. A distal portion 5116 of the elongated portion 5114 includes an image sensor 5142, a lens 5144, a lens barrel 5146, a protective window 5147, light source (e.g., LED light emitting elements) 5150, a processor 5160, a sensor substrate 5180, and a light source substrate 5190. Distal portion 5116 of the elongated portion terminates in a distal end 5118.

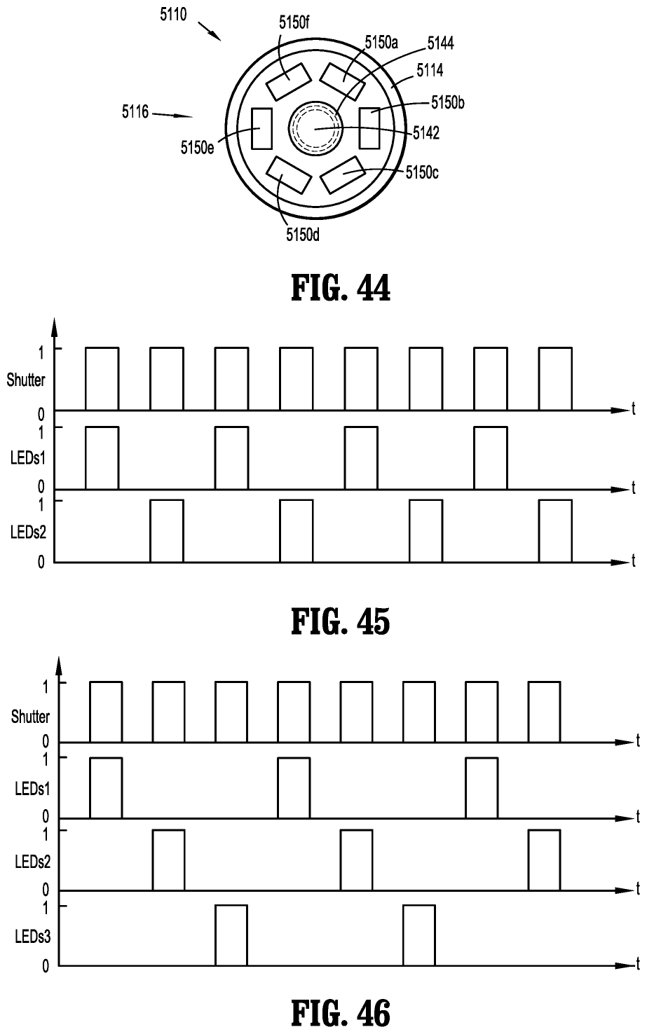

In the embodiment illustrated in FIG. 44, light source 5150 includes six LED light emitting elements 5150a, 5150b, 5150c, 5150d, 5150e, and 5150f; while six lights emitting elements are illustrated, it is contemplated and within the scope of the present disclosure for more or fewer LED light emitting elements to be used in connection with endoscope 5110. Additionally, LED light emitting elements 5150a, 5150b, 5150c, 5150d, 5150e, and 5150f may be any combination of white, red, green and blue light emitting elements, for example.

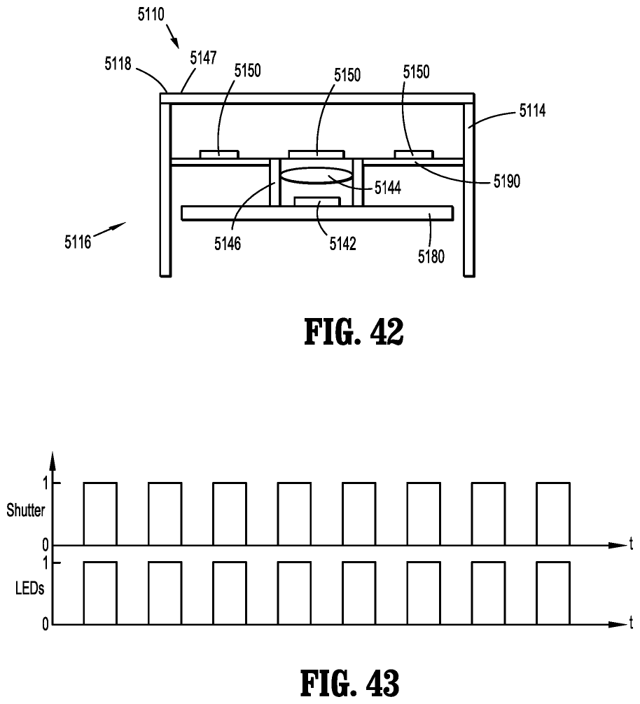

With particular reference to FIGS. 42 and 44, LED light emitting elements 5150a-5150f of light source 5150 are positioned radially outwardly of lens 5144, and are engaged with (e.g., affixed to) light source substrate 5190, which is disposed distally of lens 5144. Sensor substrate 5180 is positioned proximally of lens 5144, and lens barrel 5146 extends distally from image sensor 5142 and from sensor substrate 5180. Lens 5144 is disposed within lens barrel 5146. Image sensor 5142 is engaged with or connected to (e.g., affixed to) sensor substrate 5180. The processor 5160 is engaged with or connected to light source 5150 and is in electrical communication with the controller 5170. The controller 5170 of endoscope 5110 is similar to the controller 1170 discussed above with reference to endoscope 1110, as the controller 5170 of endoscope 5110 is configured to control the light source 5150 and the image sensor 5142.

Endoscope 5110 is configured to help ensure a minimal amount of heat is produced by light source 5150. Specifically, light source 5150 is configured to only provide illumination (and thus produce heat) when the exposure of image sensor 5142 is open. In such embodiments, the controller 5170 sends current (and thus radiance) to light source 5150 only when the exposure of image sensor 5142 is open, thus reducing the total amount of heat produced. In disclosed embodiments, image sensor 5142 is set with a constant exposure time, which is determined in part by the frequency of shooting an image, and the quality of the images. It is envisioned that the exposure time may be from about 1/100 seconds to about 1 second, and that the interval between exposures equals the reciprocal of the frame frequency minus exposure time and it may be from about 1/100 seconds to about 1 second. Light source 5150 is configured to only provide illumination (and thus produce heat) when the exposure of image sensor 5142 is open.

Additionally, endoscope 5110 is configured such that only some of the LED light emitting elements 5150a-5150f of light source 5150 are on and configured to illuminate tissue while the image sensor 5142 takes a first picture, while other LED light emitting elements 5150a-5150f are turned off while the image sensor 5142 takes the first picture. Further, the LED light emitting elements 5150a-5150f that were off while the image sensor 5142 took the first picture are on while the image sensor 5142 takes a second picture, and the LED light emitting elements 5150a-5150f that were on while the image sensor 5142 took the first picture are off while the image sensor 5142 takes the second picture. Thus, LED light emitting elements 5150a-5150f take turns illuminating tissue which helps all of the LED light emitting elements 5150a-5150f have more time to cool down, resulting in a relatively low temperature of the distal tip 5118 of the endoscope 5110. Therefore, the present disclosure includes a method of illuminating tissue by alternating the use of LED light emitting elements 5150a-5150f, as further discussed below.

It is envisioned that one, two, three, four or five LED light emitting elements 5150a-5150f are illuminated as a group or set. More particularly, and with reference to FIGS. 43, 45 and 46, various groupings of LED light emitting elements 5150a-5150f are shown. For reference, FIG. 43 is a graph illustrating when all of the LED light emitting elements 5150a-5150f are in a single group. Here, every time the shutter or exposure is open (indicated as number 1 on the graph), each LED light emitting element 5150a-5150f is on (indicated as number 1 on the graph).

In FIG. 45, the LED light emitting elements 5150a-5150f are divided into two groups, labeled LEDs 1 and LEDs2, with each group including half (or three) of the LED light emitting elements. It is envisioned that the first group (LEDs1) includes LED light emitting elements 5150a, 5150c and 5150e, and the second group (LEDs2) includes LED light emitting elements 5150b, 5150d and 5150f Here, the first time the shutter is open, LEDs1 is illuminated, and LEDs2 is off (indicted as number 0 on the graph). The second time the shutter is open, LEDs1 is off, and LEDs2 is illuminated. This alternating illumination of the LED light emitting elements of groups 1 and 2 continues for the duration of the imaging. Accordingly, each LED light emitting element 5150a-5150f is on about half of the time that the shutter is open.

In FIG. 46, the LED light emitting elements 5150a-5150f are grouped into three groups, labeled LEDs1, LEDs2 and LEDs3, with each group including a third (or two) of the LED light emitting elements. It is envisioned that the first group (LEDs1) includes LED light emitting elements 5150a and 5150d, the second group (LEDs2) includes LED light emitting elements 5150b and 5150e, and the third group (LEDs3) includes LED light emitting elements 5150c and 5150f. Here, the first time the shutter is open, LEDs1 is illuminated, and LEDs2 and LEDs3 are off. The second time the shutter is open, LEDs1 and LEDs3 are off, and LEDs2 is illuminated. The third time the shutter is open, LEDs1 and LEDs2 are off, and LEDs3 is illuminated. This alternating illumination of the LED light emitting elements of groups 1, 2 and 3 continues for the duration of the imaging. Accordingly, each LED light emitting element 5150a-5150f is on about one third of the time that the shutter is open.

Accordingly, since each LED light emitting element 5150a-5150f is only on and producing heat for a fraction of the time tissue is being illuminated, only a fraction of heat is being produced when compared to traditional illumination techniques by the same total number of LED light emitting element. This reduction in the amount of heat used by each LED light emitting element 5150a-5150f may improve the image quality, increase the life of endoscope 5110, and reduce the chances of unnecessarily heating tissue with distal portion 5116 of endoscope 5110.

It will be understood that various modifications may be made to the embodiments described herein. Therefore, the above description should not be construed as limiting, but merely as exemplifications of various embodiments. Those skilled in the art will envision other modifications within the scope and spirit of the claims appended thereto.

1. An endoscope comprising:
a handle;
an elongated body extending distally from the handle, the elongated body including a distal portion terminating at a distal end;
an image sensor disposed within the distal portion of the elongated body and configured to capture a plurality of images;
a lens disposed adjacent the distal end of the elongated body;
a light source disposed within the distal portion of the elongated body and configured to illuminate tissue;
a processor disposed in electrical communication with the image sensor and the light source, the processor configured to analyze at least one characteristic of a first image of the plurality of images captured by the image sensor; and
a controller disposed in electrical communication with the processor, the controller configured to supply current to the light source based on the at least one characteristic of the first image of the plurality of images analyzed by the processor.

2. The endoscope according to claim 1, wherein the controller is configured to supply current to the light source based at least one characteristic of a second image of the plurality of images captured by the image sensor.

3. The endoscope according to claim 1, wherein the at least one characteristic of the first image is average gray scale.

4. The endoscope according to claim 2, wherein the at least one characteristic of the first image is average gray scale and wherein the at least one characteristic of the second image is average gray scale.

5. The endoscope according to claim 1, wherein the light source is configured to illuminate tissue only when an exposure of the image sensor is open.

6. The endoscope according to claim 5, wherein the exposure of the image sensor is configured to be open from about 1/100 seconds to about 1 second before the exposure is closed.

7. The endoscope according to claim 1, wherein the light source includes one or more light-emitting diodes.

8. The endoscope according to claim 1, wherein the light source includes four light-emitting diodes.

9. The endoscope according to claim 1, wherein the controller is disposed within the handle.

10. The endoscope according to claim 1, further comprising a high thermal conductivity layer disposed adjacent the distal end of the elongated body.

11. The endoscope according to claim 10, wherein the high thermal conductivity layer includes graphene.

12. The endoscope according to claim 10, wherein the high thermal conductivity layer has a thickness of from about 0.02 mm to about 0.5 mm.

13. A method of using an endoscope, the method comprising:
positioning a distal end of an endoscope adjacent tissue, the endoscope including:
a handle;
an elongated body extending distally from the handle, the elongated body including a distal portion terminating at a distal end;
an image sensor disposed within the distal portion of the elongated body;
a light source disposed within the distal portion of the elongated body;
a processor disposed in electrical communication with the image sensor and the light source; and
a controller disposed in electrical communication with the processor;
capturing a first image using the image sensor;
analyzing the first image using the processor to obtain at least one characteristic;
supplying a first amount of current to the light source from the controller based on the at least one characteristic of the first image;
capturing a second image using the image sensor;
analyzing the second image using the processor to obtain at least one characteristic of the second image; and
supplying a second amount of current to the light source from the controller based on the at least one characteristic of the second image.

14. The method according to claim 13, wherein the first amount of current is different from the second amount of current.

15. The method according to claim 13, wherein supplying a first amount of current to the light source from the controller is based on average gray scale of the first image.

16. The method according to claim 13, wherein supplying a second amount of current to the light source from the controller is based on average gray scale of the second image.

17. The method according to claim 13, further comprising opening and closing an exposure of the image sensor.

18. The method according to claim 17, further comprising illuminating tissue using the light source while the exposure of the image sensor is open.

19. The method according to claim 17, further comprising closing an exposure of the image sensor after the exposure has been open from about 1/100 seconds to about 1 second.

20-59. (canceled)
